(19) United States
(12) Patent Application Publication
(10) Pub. No.: US 2026/0356790 A1
(43) Pub. Date: Nov. 12, 2026

(54) PROCESS FOR PREPARING ALKYL METHACRYLATES

(71) Applicants: Dow Global Technologies LLC, Midland, MI (US); Rohm and Haas Company, Collegeville, PA (US)

(72) Inventors: William G. Worley, Missouri City, TX (US); Kirk W. Limbach, Dresher, PA (US)

(21) Appl. No.: 19/125,957

(22) PCT Filed: Nov. 20, 2023
(86) PCT No.: PCT/US2023/080434

§ 371 Date: Apr. 30, 2025

Related U.S. Application Data
(60) Provisional application No. 63/431,039, filed on Dec. 8, 2022.

(51) Int. Cl. C07C 67/44; C07C 67/54

(52) U.S. Cl. CPC C07C 67/44; C07C 67/54

(57) ABSTRACT

Provided is a process for preparing an alkyl methacrylate comprising reacting methacrolein with an alkyl alcohol in an oxidative esterification reaction (OER) system in the presence of at least one inhibitor, an oxygen-containing gas, and a noble metal-containing catalyst comprising to produce an OER product stream comprising an alkyl methacrylate, an alkyl isobutyrate in an amount ranging from 0.1 to 5000 ppm, and at least one Michael addition product in an amount ranging from 0.01 to 5 weight % based on the total weight of the OER product stream. The OER product stream is fed to an alcohol recovery distillation column to provide an overhead stream comprising alkyl alcohol and methacrolein and a bottoms stream comprising alkyl methacrylate, alkyl isobutyrate, acetals and/or hemiacetals of methacrolein. The bottoms stream of the alcohol recovery distillation column is fed to an acetal hydrolysis reactor and a phase separator.

### FIELD OF THE INVENTION

This invention relates to a process for preparing alkyl methacrylates.

### BACKGROUND

The conversion of an aldehyde and alcohol in the presence of oxygen to a carboxylic ester via oxidative esterification, and in particular the conversion of methacrolein and methanol in the presence of oxygen to methyl methacrylate, has been known for many years. For example, U.S. Pat. No. 4,249,019 discloses the use of a palladium (Pd)-lead (Pb) catalyst and other catalysts for this purpose.

Typical process configurations have included slurry catalyst bubble column reactors and slurry catalyst continuous stirred tank reactors (CSTR). Slurry type reactors for this chemistry typically use a catalyst of less than 200 μm size, and U.S. Pat. No. 6,228,800 discloses the use of an egg-shell type catalyst of less than 200 μm size for slurry reactions. Issues with the use of slurry catalysts stem from catalyst attrition which may limit the life of the catalyst and make filtration of the product stream difficult. According to CN1931824, these problems can be addressed through the use of a larger size catalyst charged to a fixed bed reactor. However, as noted in U.S. Patent Application Publication No. 2016/0251301, the use of larger catalyst particles leads to a reduced space-time yield and other potential disadvantages.

Fixed bed technology with larger catalyst particles has been implemented in U.S. Pat. No. 4,520,125, which discloses the use of a 4 mm diameter catalyst in a fixed bed system. The reactor feed in that case was relatively dilute, as it is in more recent discussions of fixed bed technology for this chemistry such as U.S. Patent Application Publication No. 2016/0251301 and U.S. Patent Application Publication No. 2016/0280628.

In commercial production facilities, the oxidative esterification reactors are followed by a separation section consisting of distillation columns to purify the product and recycle dewatered and otherwise purified unreacted reactants (see, e.g., U.S. Pat. No. 5,969,178) where the product and recycle often constitute the majority of the product stream. In part, this is because methanol is typically provided to the oxidative esterification reactor in excess to maximize the conversion of valuable methacrolein (see, e.g., U.S. Pat. No. 7,326,806).

Feed concentration of methacrolein into the oxidative esterification reactor varies in the literature from very low (see, e.g., U.S. Pat. No. 5,892,102) to around 35 wt % (see, e.g., U.S. Pat. No. 8,461,373). Methanol is typically the major constituent of the feed and the recycle stream that returns to the oxidative esterification reactor from the downstream separations section.

Catalysts for this chemistry have included various noble metals such as palladium-based catalysts including palladium-lead catalyst (see, e.g., U.S. Pat. No. 4,249,019) and gold-based or gold-containing catalysts (see, e.g., U.S. Pat. Nos. 7,326,806 and 8,461,373).

It is desirable to maximize selectivity and reduce the formation of all byproducts. In particular, byproduct methyl isobutyrate (MIB) is critical to reduce because it is difficult to separate from the product MMA and is undesirable in the product.

### STATEMENT OF INVENTION

One aspect of the invention provides a process for preparing an alkyl methacrylate comprising reacting methacrolein with an alkyl alcohol in an oxidative esterification reaction (OER) system in the presence of at least one inhibitor, an oxygen-containing gas, and a noble metal-containing catalyst comprising to produce an OER product stream comprising an alkyl methacrylate, an alkyl isobutyrate in an amount ranging from 0.1 to 5000 ppm, and at least one Michael addition product in an amount ranging from 0.01 to 5 weight % based on the total weight of the OER product stream. The OER product stream is fed to an alcohol recovery distillation column to provide an overhead stream comprising alkyl alcohol and methacrolein and a bottoms stream comprising alkyl methacrylate, alkyl isobutyrate, acetals and/or hemiacetals of methacrolein. The bottoms stream of the alcohol recovery distillation column is fed to an acetal hydrolysis reactor and a phase separator.

### DETAILED DESCRIPTION

All percentage compositions are weight percentages (wt %), all amounts provided in terms of parts per million (ppm) are on the basis of weight, and all temperatures are in °C, unless otherwise indicated. Averages are arithmetic averages unless otherwise indicated. An “average concentration” is the arithmetic average of the concentration entering a region and the concentration exiting the region, where the region is an individual reactor, a reactor system, or a zone within a reactor or reactor system. An “average ratio” is the ratio of the average concentration of one component relative to the average concentration of another component. For example, the average ratio of alcohol to methacrolein in a reactor system is calculated by dividing the average concentration of alcohol entering and exiting the reactor system by the average concentration of methacrolein entering and exiting the reactor system. A noble metal is any of gold, platinum, iridium, osmium, silver, palladium, rhodium and ruthenium. More than one noble metal may be present in the catalyst, in which case the limits apply to the total of all noble metals.

The “catalyst center” is the centroid of the catalyst particle, i.e., the mean position of all points in all coordinate directions. A diameter is any linear dimension passing through the catalyst center and the average diameter is the arithmetic mean of all possible diameters. The aspect ratio is the ratio of the longest to the shortest diameters.

A reactor system refers to one or more reactors where a designated reaction takes place. For example, the oxidative esterification of methacrolein to produce an alkyl methacrylate may be the designated reaction that takes place in the reactor system. The reactor system may comprise a single reactor or a plurality of reactors. Additionally, the reactor system may be subdivided into multiple zones, i.e., a multizone reactor system. Zones may be defined by physical separation, such as by walls or barriers that define separate areas, or by differences in the reaction conditions, such as, for example, pressure, temperature, composition or concentration of the catalyst, reactants, or other reaction components such as inert materials, pH modifiers, etc. For example, the reactor system may comprise a single reactor comprising a single zone, a single reactor comprising multiple zones, multiple reactors comprising a single zone in each reactor, multiple reactors where one or more reactors has a single zone and one or more reactors that comprise multiple zones, or multiple reactors each comprising multiple zones. By definition, a reactor system comprising multiple reactors would be considered a multizone reactor system. An example of a multizone reactor may be a continuous tubular reactor comprising multiple zones, including one or more mixing zones, a cooling zone, and one or more catalyst zones where the reaction takes place. Another example of a multizone single reactor may be a stirred bed reactor comprising internal walls containing the catalyst that defines a catalyst zone through which liquid reactants are circulated, and a feed/removal zone outside of the catalyst zone where the reactants enter the reactor and products exit the reactor. When referring to the average concentration or any ratio of the reactor system, the average concentration or ratio is calculated based on what enters the reactor system and what exits the reactor system.

One aspect of the present invention relates to a process for producing an alkyl methacrylate by the oxidative esterification of methacrolein in the presence of an alkyl alcohol, at least one polymerization inhibitor, and an oxygen-containing gas. The process is performed in an oxidative esterification reactor system (“OER system”) comprising a noble-metal catalyst.

The OER reactor system may comprise a single reactor or a plurality of reactors. Additionally, the reactor system may be subdivided into multiple zones, i.e., a multizone reactor system. Zones may be defined by physical separation, such as by walls or barriers that define separate areas, or by differences in the reaction conditions, such as, for example, pressure, temperature, composition or concentration of the catalyst, reactants, or other reaction components such as inert materials, pH modifiers, etc. For example, the reactor system may comprise a single reactor comprising a single zone, a single reactor comprising multiple zones, multiple reactors comprising a single zone in each reactor, multiple reactors where one or more reactors has a single zone and one or more reactors that comprise multiple zones, or multiple reactors each comprising multiple zones. By definition, a reactor system comprising multiple reactors would be considered a multizone reactor system. An example of a multizone reactor may be a continuous tubular reactor comprising multiple zones, including one or more mixing zones, a cooling zone, and one or more catalyst zones where the reaction takes place. Another example of a multizone single reactor may be a stirred bed reactor comprising internal walls containing the catalyst that defines a catalyst zone through which liquid reactants are circulated, and a feed/removal zone outside of the catalyst zone where the reactants enter the reactor and products exit the reactor. When referring to the average concentration or any ratio of the reactor system, the average concentration or ratio is calculated based on what enters the reactor system and what exits the reactor system.

The reactor system may comprise a reactor configured as a fluidized bed reactor, a fixed bed reactor, a trickle bed reactor, a packed bubble column reactor, or a stirred bed reactor. Preferably, the reactor system comprises a packed bubble column reactor.

The catalyst may be present in the form of a slurry or a fixed bed depending on the reactor in which the catalyst is present. For example, a slurry catalyst can be used in a stirred bed reactor or a fluidized bed reactor, whereas a fixed bed catalyst can be used in a fixed bed reactor, trickle bed reactor, or a packed bubble column reactor. Preferably, the reactor is in the form of a fixed bed reactor.

The size of the catalyst can be selected based on the type of reactor. For example, a slurry catalyst may have an average particle diameter less than 200 μm, such as, for example, from 10 μm to 200 μm. A fixed bed catalyst may have an average particle diameter 200 μm or greater, such as, for example, from 200 μm to 30 mm. Preferably, the average diameter of the catalyst particle is at least 200 μm, more preferably at least 400 μm, even more preferably at least 600 μm, and still more preferably at least 800 μm; preferably no more than 30 mm, more preferably no more than 20 mm, and even more preferably no more than 10 mm.

The noble metal-containing catalyst preferably comprises a noble metal selected from gold and palladium, more preferably gold. Preferably, the noble metal is in the form of particles. In a preferred embodiment, the noble metal-containing catalyst comprises gold particles having an average diameter of less than 12 nm, preferably less than 10 nm, and more preferably less than 8 nm. The standard deviation of the average diameter of the gold particles is +/−4 nm, preferably +/−2.5 nm, and more preferably +/−2 nm. As used herein, the standard deviation is calculated by the following equation:

$$standard deviation = \sqrt{\frac{{\sum }_{i = 1}^{n} \vert x - \overline{x} \vert}{n}}$$

where x is the size of each particle, x̅ is the mean of the n number of particles, and n is at least 500.

The noble metal-containing catalyst may further comprise particles of at least one metal oxide. Preferably, the metal of the at least one metal oxide is selected from aluminum, titanium, lanthanides, zirconium, alkali metals, alkaline earth metals, nickel, cobalt, zinc, magnesium, tellurium, antimony, rhenium, tungsten, and bismuth. Preferably, the metal of the at least one metal oxide is selected from nickel and titanium.

The particles of at least one metal oxide preferably have an average diameter of less than 5 times the average diameter of the noble metal particles, more preferably an average diameter of less than 4 times the average diameter of the noble metal particles, even more preferably an average particle diameter of less than 3 times the average diameter of the noble metal particles, still more preferably an average particle diameter of less than 2 times the average diameter of the noble metal particles, and yet more preferably an average particle diameter of less than 1.5 times the average diameter of the noble metal particles. Preferably, the particles of at least one metal oxide have an average diameter at least the half the average diameter of the noble metal particles, and more preferably at least the same as the average diameter of the noble metal particles

The amount by weight of the noble metal particles with respect to the amount of the particles of at least one metal oxide may range from 1:1 to 1:20. Preferably, the weight ratio of noble metal particles to particles of at least one metal oxide ranges from 1:2 to 1:15, more preferably from 1:3 to 1:10, even more preferably from 1:4 to 1:9, and still more preferably from 1:5 to 1:8.

Preferably, the noble metal particles are evenly distributed among the particles of at least one metal oxide. As used herein, the term “evenly distributed” means the noble metal particles are randomly dispersed among the particles of at least one metal oxide with substantially no agglomeration of the noble metal particles. Preferably, at least 80% of the total number of the noble metal particles are present in a particle having an average diameter less than 12 nm. More preferably, at least 90% of the total number of the noble metal particles are present in a particle having an average diameter less than 12 nm. Even more preferably, at least 95% of the total number of noble metal particles are present in a particle having an average diameter less than 12 nm.

Preferably, at least 75% of the noble metal particles by number of noble metal particles are within at least 20 nm of a metal oxide particle. As used herein, the phrase “within at least X nm” means that an edge of a noble metal particle is within X nm of an edge of the metal oxide particle nearest the noble metal particle. Preferably, at least 75% of the noble metal particles are within at least 15 nm of a metal oxide particle, more preferably within at least 12 nm of a metal oxide particle, and even more preferably within at least 10 nm of a metal oxide particle.

More preferably, at least 75% of the noble metal particles by number of the noble metal particles are within at least 20 nm of two metal oxide particles, i.e., an edge of the noble metal particle is within at least 20 nm of an edge of the two metal oxide particles nearest the noble metal particle. Preferably, at least 75% of the noble metal particles are within at least 15 nm of two metal oxide particles, more preferably within at least 12 nm of two metal oxide particles, and even more preferably within at least 10 nm of two metal oxide particles.

Even more preferably, at least 75% of the noble metal particles by number of the noble metal particles are within at least 20 nm of at least three metal oxide particles, i.e., an edge of the noble metal particle is within at least 20 nm of an edge of at least the three metal oxide particles nearest the noble metal particle. Preferably, at least 75% of the noble metal particles are within at least 15 nm of at least three metal oxide particles, more preferably within at least 12 nm of at least three metal oxide nanoparticles, and even more preferably within at least 10 nm of at least three metal oxide particles.

The noble metal particles in the catalyst may be disposed on a surface of a support material. Preferably, the support material is a particle of an oxide material; preferably γ-, δ-, or θ-alumina, silica, magnesia, titania, zirconia, hafnia, vanadia, niobium oxide, tantalum oxide, ceria, yttria, lanthanum oxide or a combination thereof. Preferably, in portions of the catalyst comprising the noble metal, the support has a surface area greater than 10 m²/g, preferably greater than 30 m²/g, preferably greater than 50 m²/g, preferably greater than 100 m²/g, preferably greater than 120 m²/g. In portions of the catalyst which comprise little or no noble metal, the support may have a surface area less than 50 m²/g, preferably less than 20 m²/g. The average diameter of the support and the average diameter of the final catalyst particle are not significantly different.

Preferably, the aspect ratio of the catalyst particle is no more than 10:1, preferably no more than 5:1, preferably no more than 3:1, preferably no more than 2:1, preferably no more than 1.5:1, preferably no more than 1.1:1. Preferred shapes for the catalyst particle include spheres, cylinders, rectangular solids, rings, multi-lobed shapes (e.g., cloverleaf cross section), shapes having multiple holes and “wagon wheels;” preferably spheres. Irregular shapes may also be used.

The noble metal particles can be dispersed throughout the catalyst or have varying concentration densities, such as, for example, a gradient concentration or layered structure. Preferably, at least 90 wt % of the noble metal particles are in the outer 70% of catalyst volume (i.e., the volume of an average catalyst particle), preferably the outer 60% of catalyst volume, preferably the outer 50%, preferably the outer 40%, preferably the outer 35%, preferably in the outer 30%, preferably in the outer 25%. Preferably, the outer volume of any particle shape is calculated for a volume having a constant distance from its inner surface to its outer surface (the surface of the particle), measured along a line perpendicular to the outer surface. For example, for a spherical particle the outer x % of volume is a spherical shell whose outer surface is the surface of the particle and whose volume is x % of the volume of the entire sphere. Preferably, at least 95 wt % of the noble metal is in the outer volume of the catalyst, preferably at least 97 wt %, preferably at least 99 wt %. Preferably, at least 90 wt % (preferably at least 95 wt %, preferably at least 97 wt %, preferably at least 99 wt %) of the noble metal(s) is within a distance from the surface that is no more than 30% of the catalyst diameter, preferably no more than 25%, preferably no more than 20%, preferably no more than 15%, preferably no more than 10%, preferably no more than 8%. Distance from the surface is measured along a line which is perpendicular to the surface.

Preferably, the catalyst comprises gold particles and particles of at least one metal oxide, wherein the metal of the at least one metal oxide is selected from titanium and nickel, on a support material comprising silica. Preferably, the gold particles and particles of at least one metal oxide form an eggshell structure on the support particles. The eggshell layer may have a thickness of 500 microns or less, preferably 250 microns or less, and more preferably 100 microns or less.

Preferably, at least 0.1% by weight of the total weight of the noble metal particles are exposed on a surface of the catalyst, where the surface includes both the outer surface and pores of the catalyst. As used herein, the term “exposed” means that at least a portion of the noble metal particle is not covered by another noble metal particle or a particle of at least one metal oxide, i.e., the reactants can directly contact the gold particle. The noble metal particles may therefore be disposed within a pore of the support material and still be exposed by virtue of the reactant being able to directly contact the noble metal particle within the pore. More preferably, at least 0.25% by weight of the total weight of the noble metal particles are exposed on the surface of the catalyst, even more preferably, at least 0.5% by weight of the total weight of the noble metal particles are exposed on the surface of the catalyst, and still more preferably, at least 1% by weight of the total weight of the noble metal particles are exposed on the surface of the catalyst.

The catalyst bed may further comprise inert or acidic materials. Preferred inert or acidic materials include, e.g., alumina, clay, glass, silica carbide and quartz. Preferably, the inert or acidic materials located before and/or after the catalyst bed have an average diameter equal to or greater than that of the catalyst, preferably 1 to 30 mm; preferably at least 2 mm; preferably no greater than 30 mm, preferably no greater than 10 mm, preferably no greater than 7 mm.

Preferably, the catalyst bed in the OER system is at a pressure from 1 to 150 bar (100 to 15000 kPa). Without wishing to be limited by theory, it is believed that operating the OER system at increased pressure will lower the amount of MIB present in the product stream by increasing the amount of oxygen present in the liquid phase. Therefore, the pressure in the catalyst bed of the OER system may be at least 10 bar, more preferably at least 20 bar, even and more preferably at least 30 bar, and preferably less than 150 bar, and more preferably less than 120 bar. When the OER system comprises more than one reactor and/or zone, the pressure in each reactor and/or zone may be the same or different.

The heterogeneous noble metal-containing catalyst in the OER system may be present in an amount ranging from 0.02 kg to 2 kg of catalyst for every gram-mole of alkyl methacrylate exiting the reactor system over the course of 1 hour. Preferably, the heterogeneous noble metal-containing catalyst in the OER system is present in an amount of at least 0.02 kg to 0.5 kg of catalyst, for every gram-mole of alkyl methacrylate exiting the reactor system over the course of 1 hour. Preferably, the heterogeneous noble metal-containing catalyst in the OER system is present in an amount of less than 0.4 kg of catalyst, more preferably less than 0.3 kg of catalyst, still more preferably less than 0.25 kg of catalyst, and even more preferably less than 0.2 kg of catalyst for every gram-mole of alkyl methacrylate exiting the reactor system over the course of 1 hour.

When the noble metal-containing catalyst comprises gold, the gold may be present in an amount ranging from 0.0001 kg to 0.1 kg for every gram-mole of alkyl methacrylate exiting the reactor system over the course of 1 hour. Preferably, the gold is present in an amount of at least 0.0001 kg to 0.005 kg for every gram-mole of alkyl methacrylate exiting the reactor system over the course of 1 hour. Preferably, the gold is present in an amount less than 0.004 kg for every gram-mole of alkyl methacrylate exiting the reactor system over the course of 1 hour.

In terms of the amount of heterogeneous noble metal-containing catalyst in the OER system with respect to the amount of methacrolein entering the reactor system, at 50% conversion of methacrolein entering the OER system, the gold in the heterogeneous noble metal-containing catalyst in the OER system may be present in an amount ranging from 0.00005 to 0.05 kg of gold for every gram-mole of methacrolein entering the reactor system over the course of 1 hour. At 25% conversion of methacrolein entering the OER system, the gold in the heterogeneous noble metal-containing catalyst in the OER system may be present in an amount ranging from 0.000025 to 0.025 kg of catalyst for every gram-mole of methacrolein entering the reactor system over the course of 1 hour. At 75% conversion of methacrolein entering the OER system, the gold in the heterogeneous noble metal-containing catalyst in the OER system may be present in an amount ranging from 0.000075 to 0.075 kg of catalyst for every gram-mole of methacrolein entering the reactor system over the course of 1 hour.

In the OER reactor system, an alkyl methacrylate is produced by reacting methacrolein with an alkyl alcohol in the presence of an oxygen-containing gas. The alkyl group of the alkyl methacrylate is a straight or branched C₁ to C₁₂ alkyl group. The alkyl alcohol comprises a straight or branched alcohol comprising from 1 to 12 carbon atoms. Preferably, the alkyl alcohol is selected from the group consisting of methanol, ethanol, propanol, butanol, hexanol, 2-ethylhexanol, and octanol, in all of their isomeric forms. More preferably, the alkyl alcohol is selected from the group consisting of methanol, ethanol, butanol, and 2-ethylhexanol. Even more preferably, the alkyl alcohol is methanol.

Preferably, the concentration of alkyl alcohol entering the OER system is greater than 32 wt % based on the total weight of alkyl alcohol and methacrolein entering the reactor system. More preferably, the concentration of alkyl alcohol entering the OER system is greater than 35 wt %, and even more preferably greater than 40 wt % based on the total weight of alkyl alcohol and methacrolein entering the reactor system. Preferably, the concentration of alkyl alcohol entering the OER system is less than 75 wt % based on the total weight of alkyl alcohol and methacrolein entering the reactor system. More preferably, the concentration of alkyl alcohol entering the OER system is less than 60 wt %, and even more preferably less than 50 wt % based on the total weight of alkyl alcohol and methacrolein entering the reactor system.

Preferably, the average concentration of alkyl alcohol in the OER system (i.e., the arithmetic average of the concentration of the alkyl alcohol entering and exiting the OER system) is greater than 70 wt % based on the average total weight of alkyl alcohol and methacrolein entering the reactor system (i.e., the arithmetic average of the total weight of methanol and methacrolein entering the OER system and the total weight of methanol and methacrolein exiting the OER system). More preferably, the average concentration of alkyl alcohol in the OER system is greater than 75 wt % based on the average total weight of alkyl alcohol and methacrolein entering and exiting the reactor system.

It is preferred that the average weight ratio of alkyl alcohol to methacrolein in the OER system ranges from 20:1 to 2:1, where the average weight ratio is based on the average concentration of alkyl alcohol entering and exiting the OER system and the average concentration of methacrolein entering and exiting the OER system.

Preferably, the liquid phase in the OER system is at a temperature from 40 to 120 °C; preferably at least 50 °C, and preferably at least 55 °C. The temperature of the liquid phase in the OER system is preferably no more than 110 °C, and preferably no more than 100 °C.

When the OER system comprises more than one reactor and/or more than one zone, the temperature in each reactor and/or zone may be the same or different. For example, a reaction mixture exiting a reactor or zone may be cooled prior to entering the next reactor or zone.

The pH in the catalyst bed may range from 2 to 10. Some catalysts may be deactivated in acidic conditions. Therefore, when the catalyst is not acid resistant, the pH in the catalyst bed is from 4 to 10; preferably at least 5, preferably at least 5.5; preferably no greater than 9, preferably no greater than 8, preferably no greater than 7.5.

To increase the pH in the reactor system, a base material may be added. The base material may comprise an Arrhenius base (i.e., a compound that dissociates in water to form hydroxide ions), a Lewis base (i.e., a compound capable of donating a pair of electrons), or a Bronsted-Lowry base (i.e., a compound capable of accepting a proton). Examples of Arrhenius bases include, but are not limited to, hydroxides of alkali and alkali earth metals. Examples of Lewis bases include, but are not limited to, amines, sulfates, and phosphines. Examples of Bronsted-Lowry bases include, but are not limited to, halides, nitrates, nitrites, chlorites, chlorates, etc. Ammonia can be either a Lewis base or a Bronsted-Lowry base.

The present inventors have discovered that high localized concentrations of base materials in the reactor system can cause the formation of unwanted Michael addition products as byproducts. Therefore, to aid in the minimization of the formation of Michael addition products, the base material is preferably mixed with at least one other material prior to entering the reactor system. Preferably, the base material is introduced at a position external to the reactor system and mixed with one or more reactants or diluents to form a base-containing stream. For example, the base material may be mixed with the alkyl alcohol, water, or a non-reactive solvent, i.e., a solvent that does not negatively impact the formation of the alkyl methacrylate in the reactor system. The position external to the reactor system may be a mixing vessel. Alternatively, the position external to the reactor may be a line through which components travel to the reactor system, such as a feed line or a recycle line, in which sufficient mixing occurs, such as by turbulent flow, baffles, jet mixer, or other mixing method.

Preferably, the amount of the base material in the base-containing stream is 50 wt % or less based on the total weight of the base-containing stream, preferably 25 wt % or less, preferably 20 wt % or less, preferably 15 wt % or less, preferably 10 wt % or less, preferably 5 wt % or less, or preferably 1 wt % or less. The base material is preferably diluted by a factor of less than 1:2, such as, less than 1:3, less than 1:4, less than 1:5, less than 1:10, less than 1:20, or less than 1:100, relative to the total weight of the base-containing stream prior to entering the reactor system. Preferably, the amount of base material added to the OER reactor system is less than 10 weight %, more preferably less than 5 weight %, and even more preferably less than 2 weight %, based on the total weight of reactants in the OER reactor system.

Preferably, the base-containing stream is sufficiently mixed to avoid localized spikes in the concentration of the base material within the base-containing stream before it is added to the reactor system. For example, it is preferred that the base-containing stream reach at least 95% degree of homogeneity, i.e., variations in the concentration of the base material deviate within +/−5% of the average concentration of base material for the base-containing stream prior to entering the reactor system. Preferably, the base-containing stream reaches 95% degree of homogeneity within 4 minutes of introduction of the base material, more preferably within 2 minutes, and even more preferably within 1 minute of introduction of the base material.

For a mixing vessel, the time required for an additive to reach a 95% degree of homogeneity is defined at Θ₉₅, which can be calculated by the method disclosed by Grenville and Nienow, The Handbook of Industrial Mixing, Pages 507-509, which gives the following expression for a stirred tank in turbulent flow:

$${θ}_{95} = 5.2 \frac{{T}^{1.5} {H}^{0.5}}{{D}^{2} {N}_{P}^{1 / 3} N}$$

where T is the tank diameter, H is the liquid height, D is the impeller diameter, N_{P} is the characteristic power number of the impeller(s), and N is the impeller speed. Similar expressions exist for static mixers, jet mixed vessels, etc.

Preferably, no base material is added to the reactor system, either internal or external to the reactor system. Preferably, when no base material is added to the reactor system, the noble metal-containing catalyst comprises an acid-resistant catalyst such as a catalyst comprised of gold and titanium-containing particles. Operating the OER system in the absence of a base material may provide several advantages. One advantage is the increased selectivity and space time yield (STY) due to lower production of Michael addition products. Another advantage is the reduction in cost due to the reduced cost to treat aqueous waste. Aqueous waste exiting an oxidative esterification process in which a base material was used can produce large quantities of inorganic salts, which can be difficult or impossible to treat with biological water treatment processes. This in turn, may require the use of other waste treatment process, such as incineration.

One example of an OER system comprises a multizone or multi-reactor system. In a first zone or reactor, the average concentration of alkyl alcohol in the first zone or reactor ranges from 50 wt % to 80 wt % based on the average total amount of alkyl alcohol and methacrolein entering and exiting the first zone or reactor. The final zone or reactor has an average alkyl alcohol concentration ranging from 80 wt % to 100 wt % based on the average total amount of alkyl alcohol and methacrolein entering and exiting the final zone or reactor. Between the first zone or reactor and the final zone or reactor, the reactor mixture may be cooled and/or additional oxygen may be added, such as, for example, by adding air to a gas phase entering the final zone or reactor.

Polymerization inhibitors are introduced into the OER system. Inhibitors can also be introduced into the process at additional locations to control unwanted polymerization. For example, inhibitors can be added to any intermediate or product streams, any phase separators, and any distillation columns present in subsequent purification operations. Suitable inhibitors include, for example, 4-hydroxy-2,2,6,6-tetramethylpiperidin-1-oxyl (4-Hydroxy-TEMPO).

The OER system produces a product stream comprising a liquid phase comprising methacrolein, the alkyl alcohol and the alkyl methacrylate and a gaseous phase comprising oxygen. The liquid phase may further comprise byproducts, e.g., Michael addition products, methacrolein acetals and hemiacetals, such as, for example, methacrolein dimethyl acetal (MDA) or methacrolein dibutyl acetal, and an isobutyrate of the alkyl alcohol, such as, for example, methyl isobutyrate (MIB) or butyl isobutyrate (BIB). Without taking steps to control its formation, alkyl isobutyrates may be present in an alkyl methacrylate product stream in amounts in excess of 1 wt % (10,000 ppm) relative to the total weight of alkyl methacrylate, methacrolein and alkyl alcohol in the product stream exiting the OER system. Alkyl isobutyrates can be difficult to separate from the alkyl methacrylate. Therefore, the present invention seeks to limit the amount of alkyl isobutyrates that are formed such that the amount of alkyl isobutyrate in the product stream ranges from 0.1 ppm to 5000 ppm, preferably from 0.1 to 4000 ppm, more preferably from 0.1 to 3000 ppm, even more preferably from 0.1 to 2500 ppm, still more preferably from 0.1 to 2000 ppm, and yet more preferably from 0.1 to 1000 ppm, based on the total weight of the product stream. Preferably, the amount of Michael products in the product stream is ranges from 0.01 to 5 weight %, more preferably from 0.01 to 3 weight %, still more preferably from 0.01 to 2 weight %, and even more preferably from 0.01 to 1 weight %, based on the total weight of the product stream. Preferably, the amount of acetals and hemiacetals of methacrolein in the product stream ranges from 0.01 to 10 weight %, more preferably from 0.01 to 5 weight %, and even more preferably from 0.01 to 3 weight %, based on the total weight of the alkyl methacrylate and acetals and hemiacetals of methacrolein in the product stream exiting the OER system.

Preferably, the concentration of alkyl alcohol in the liquid phase product stream exiting the OER system ranges from 15 wt % to 95 wt % based on the total weight of the liquid phase product stream exiting the OER system. For example, the concentration of alkyl alcohol in the liquid phase product stream exiting the OER system may be at least 20 wt %, at least 25 wt %, or at least 30 wt % based on the total weight of the liquid phase product stream exiting the OER system. Preferably, the concentration of alkyl alcohol in the liquid phase product stream exiting the OER system is less than 90 wt %, more preferably less than 80 wt %, even more preferably less than 70 wt %, still more preferably less than 60 wt %, and yet more preferably less than 50 wt % based on the total weight of the liquid phase product stream exiting the OER system.

Preferably, oxygen concentration in a gas stream exiting the OER system is at least 1 mol %, more preferably at least 2 mol %, even more preferably at least 2.5 mol %, still more preferably at least 3 mol %, yet more preferably at least 3.5 mol %, even yet more preferably at least 4 mol %, and most preferably at least 4.5 mol %, based on the total volume of the gas stream exiting the OER system. Preferably, the oxygen concentration in a gas stream exiting the OER system is no more than 7.5 mol %, preferably no more than 7.25 mol %, preferably no more than 7 mol %, based on the total amount of the gas stream exiting the OER system.

The amount of alkyl methacrylate exiting the reactor is dependent on the conversion of methacrolein in the OER system. For example, at 50% conversion of methacrolein entering the OER system, 2 moles of methacrolein would be required for every mole of alkyl methacrylate produced. In this example, the heterogeneous noble metal-containing catalyst in the OER system may be present in an amount ranging from 0.01 to 1 kg of catalyst for every gram-mole of methacrolein entering the reactor system over the course of 1 hour. At 25% conversion of methacrolein entering the OER system, 4 moles of methacrolein would be required for every mole of alkyl methacrylate produced, and the heterogeneous noble metal-containing catalyst in the OER system may be present in an amount ranging from 0.005 to 0.5 kg of catalyst for every gram-mole of methacrolein entering the reactor system over the course of 1 hour. At 75% conversion of methacrolein entering the OER system, 1.33 moles of methacrolein would be required for every mole of alkyl methacrylate produced, and the heterogeneous noble metal-containing catalyst in the OER system may be present in an amount ranging from 0.015 to 1.5 kg of catalyst for every gram-mole of methacrolein entering the reactor system over the course of 1 hour. Disregarding any external recycle streams, the OER system preferably exhibits at least 25% conversion of methacrolein to alkyl methacrylate, more preferably at least 35% conversion, and even more preferably at least 40% conversion of methacrolein to alkyl methacrylate in the OER system. Addition of an external recycle stream that recycles unreacted methacrolein to the OER system can also be used to improve the overall conversion efficiency of the process.

The product stream from the OER system is preferably subjected to at least one distillation and at least one phase separation to purify and recover components within the product stream. For example, the product stream contains unreacted methacrolein and alkyl alcohol that can be separated and returned to the OER system. Acetals and hemiacetals of methacrolein are preferably subjected to a hydrolysis reaction to recover additional methacrolein and alkyl alcohol. Michael addition products and alkyl isobutyrates present in the product stream are preferably removed.

Preferably, the product stream is fed to an alcohol recovery distillation column which provides an overhead stream rich in alkyl alcohol and methacrolein; preferably this stream is recycled back to the OER system. Some hydrolysis of the acetals and hemiacetals of methacrolein may occur in the alcohol recovery distillation column, allowing the recovery of additional alkyl alcohol and methacrolein in the alcohol recovery distillation column.

The bottoms stream from the alkyl alcohol recovery distillation column comprises the alkyl methacrylate, an isobutyrate of the alkyl alcohol, methacrylic acid, salts and water. The bottoms stream further comprises acetals and hemiacetals of methacrolein that were not hydrolyzed in the alcohol recovery distillation column. In one embodiment, the bottoms stream from the alkyl alcohol recovery distillation column is sent to an acetal hydrolysis reactor for additional hydrolysis of the acetals and hemiacetals of methacrolein followed by phase separation to separate the organic phase from the aqueous phase. In an alternative embodiment, the acetals and hemiacetals of methacrolein may be hydrolyzed in a separate acetal hydrolysis reactor following a phase separation of the alkyl alcohol recovery bottoms stream. It may be necessary to add water to the organic phase to ensure that there is sufficient water for the methacrolein dialkyl acetal hydrolysis; these amounts may be determined from the composition of the organic phase. An acid stream may also be added to the hydrolysis reactor to ensure adequate methacrolein dialkyl acetal removal. Preferably, the amount of acetals and hemiacetals of methacrolein exiting the acetal hydrolysis reactor and the phase separator ranges from 0.01 to 100 ppm, more preferably from 0.01 to 25 ppm, and even more preferably from 0.01 to 5 ppm based on the total weight of the stream exiting the acetal hydrolysis reactor and the phase separator.

In either embodiment, the stream that has been subjected to hydrolysis in the acetal reactor and the phase separator is then sent to a heavies removal column to remove Michael addition products. Preferably, the overhead stream of the heavies removal column comprises 0.01 to 1 weight %, more preferably from 0.01 to 0.5 weight %, and even more preferably from 0.01 to 0.25 weight % of Michael addition products based on the total weight of the overhead stream of the heavies removal column.

The overhead stream of the heavies removal column is then sent to an alkyl isobutyrate removal column to further reduce the amount of the alkyl isobutyrate in the product stream. Preferably, the amount of alkyl isobutyrate in the bottoms stream exiting the alkyl isobutyrate column ranges from 0.01 to 800 ppm, more preferably from 0.01 to 600 ppm, and even more preferably from 0.01 to 400 ppm based on the total weight of the bottoms stream exiting the alkyl isobutyrate column.

The bottoms stream of the alkyl isobutyrate column may be sent to an alkyl methacrylate product column to further purify the alkyl methacrylate. For example, process inhibitors which may have been added during any of the distillation or phase separation processes, may be removed and recycled.

What is claimed is:

1. A process for preparing an alkyl methacrylate comprising:
reacting methacrolein with an alkyl alcohol in an oxidative esterification reaction (OER) system in the presence of at least one inhibitor, an oxygen-containing gas, and a noble metal-containing catalyst comprising to produce an OER product stream comprising an alkyl methacrylate, an alkyl isobutyrate in an amount ranging from 0.1 to 5000 ppm, and at least one Michael addition product in an amount ranging from 0.01 to 5 weight % based on the total weight of the OER product stream;
feeding the OER product stream to an alcohol recovery distillation column to provide an overhead stream comprising alkyl alcohol and methacrolein and a bottoms stream comprising alkyl methacrylate, alkyl isobutyrate, acetals and/or hemiacetals of methacrolein; and
feeding the bottoms stream of the alcohol recovery distillation column to an acetal hydrolysis reactor and a phase separator.

2. The process of claim 1, wherein the alkyl group of the alkyl methacrylate is a straight or branched C₁ to C₁₂ alkyl group and the alkyl group of the alkyl alcohol is a straight or branched alcohol comprising from 1 to 12 carbon atoms.

3. The process of claim 2, wherein the alkyl alcohol is selected from the group consisting of methanol, ethanol, propanol, butanol, hexanol, 2-ethylhexanol, and octanol, in all of their isomeric forms.

4. The process of claim 1, wherein feeding the bottoms stream of the alcohol recovery distillation column to an acetal hydrolysis reactor and a phase separator comprises feeding the bottoms stream of the alcohol recovery distillation column to the acetal hydrolysis reactor and then to a phase separator to produce an organic phase and an aqueous phase, wherein the organic phase comprises an amount of acetals and/or hemiacetals of methacrolein ranging from 0.01 ppm to 100 ppm based on a total weight of a stream exiting the acetal hydrolysis reactor and the phase separator.

5. The process of claim 1, wherein feeding the bottoms stream of the alcohol recovery distillation column to an acetal hydrolysis reactor and a phase separator comprises feeding the bottoms stream of the alcohol recovery distillation column to a phase separator to produce an organic phase and an aqueous phase, and feeding the organic phase to the acetal hydrolysis reactor to produce a stream comprising acetals and/or hemiacetals of methacrolein in an amount ranging from 0.01 ppm to 100 ppm based on the total weight of a stream exiting the acetal hydrolysis reactor and the phase separator.

6. The process of claim 4, further comprising feeding the stream exiting the acetal hydrolysis reactor and the phase separator to a heavies removal column to produce an overhead stream comprising 0.01 to 1 weight % of the at least one Michael addition products based on the total weight of the overhead stream.

7. The process of claim 6, further comprising feeding the overhead stream of the heavies removal column to an alkyl isobutyrate removal column, wherein the amount of alkyl isobutyrate in a bottoms stream exiting the alkyl isobutyrate column ranges from 0.01 to 800 ppm based on the total weight of the bottoms stream exiting the alkyl isobutyrate column.

8. The process of claim 7, further comprising feeding the bottoms stream of the alkyl isobutyrate removal column to an alkyl methacrylate product column to reduce an amount of polymerization inhibitors present in the bottoms stream.

9. The process of claim 1, wherein the alkyl alcohol comprises methanol and the alkyl methacrylate comprises methyl methacrylate.

10. The process of claim 1, wherein the noble metal-containing catalyst comprises gold.

11. The process of claim 10, wherein the gold is in the form of gold particles have an average diameter of less than 12 nm.

12. The process of claim 11, wherein the noble metal-containing catalyst further comprises particles of at least one metal oxide, wherein the metal of the at least one metal oxide is nickel or titanium.

13. The process of claim 12, wherein the particles of at least one metal oxide preferably have an average diameter of less than 5 times the average diameter of the gold particles.

14. The process of claim 12, wherein at least 75% of the gold particles are within at least 20 nm of a particle of at least one metal oxide.

15. The process of claim 1, wherein the noble metal-containing catalyst has an average particle diameter ranging from 200 μm to 30 mm.
